(19) United States
(12) Patent Application Publication
(10) Pub. No.: US 2022/0348538 A1
(43) Pub. Date: Nov. 3, 2022

(54) PROCESS OF PREPARING 1,1'-DISULFANDIYLBIS(4-FLUORO-2-METHYL-5-NITROBENZOL)

(71) Applicant: Bayer Aktiengesellschaft, Leveerkusen (DE)

(72) Inventor: Thomas HIMMLER, Odenthal (DE)

(21) Appl. No.: 17/763,034

(22) PCT Filed: Oct. 13, 2020
(86) PCT No.: PCT/EP2020/078703

§ 371 Date: Mar. 23, 2022

(30) Foreign Application Priority Data
Oct. 16, 2019 (EP) 19203479.1

(51) Int. Cl. C07C 319/24

(52) U.S. Cl. CPC C07C 319/24

(57) ABSTRACT

The present invention relates to a process for preparing 1,1′-disulfanediylbis(4-fluoro-2-methyl-5-nitrobenzene)

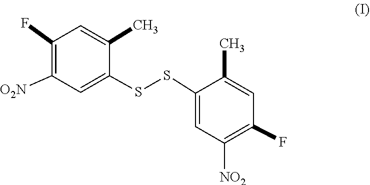

which serves as an intermediate for the preparation of insecticidally, acaricidally and nematicidally active phenyl sulfoxides.

The present invention relates to a novel process for preparing 1,1′-disulfanediylbis(4-fluoro-2-methyl-5-nitrobenzene) of formula (I).

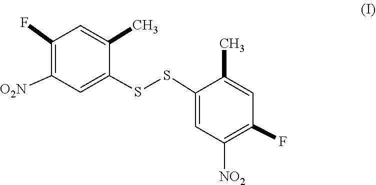

1,1′-disulfanediylbis(4-fluoro-2-methyl-5-nitrobenzene) of formula (I) is an important intermediate for the preparation of agricultural active agents and pharmaceutical active agents (cf. for example WO 2014/090913).

The preparation of 1,1′-disulfanediylbis(4-fluoro-2-methyl-5-nitrobenzene) (formula (I), CAS No. 1613615-87-4) is already known. It is for example possible to nitrate 3-fluorotoluene to give 2-fluoro-4-methylnitrobenzene of formula (II) (CAS No. 446-34-4) (cf. for example U.S. Pat. No. 4,146,625), to subsequently sulfochlorinate the nitro compound of formula (II) so as to obtain 4-fluoro-2-methyl-5-nitrobenzenesulfonyl chloride of formula (III) (CAS No. 1158953-95-7) and finally to reduce the sulfonyl chloride of formula (III) to give 1,1′-disulfanediylbis(4-fluoro-2-methyl-5-nitrobenzene) of formula (I) (see scheme 1).

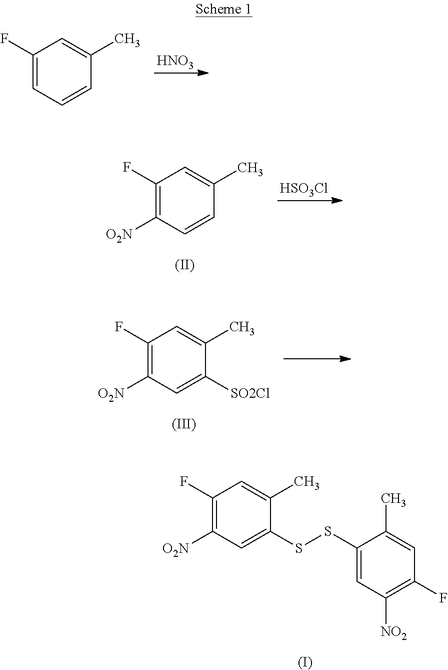

However, due to a very poor selectivity and hence also very poor yield in the nitration (only around 25% selectivity for the target product (II), the main product is 4-fluoro-2-methylnitrobenzene of formula (IV) (CAS No. 446-33-3); cf. for example U.S. Pat. No. 4,146,625; scheme 2), this synthesis is uneconomical, produces large amounts of waste and thus is unusable for a commercial industrial process.

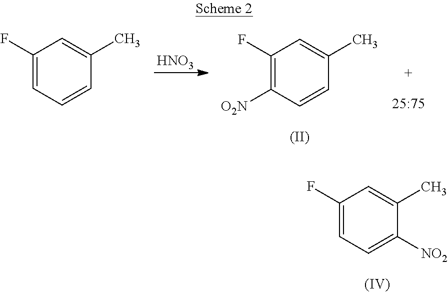

A further route for preparing the intermediate compound (III) is that of reducing the nitro compound of formula (IV) and nitrating the 4-fluoro-2-methylaniline of formula (V) (CAS No. 452-71-1) thus obtained, possibly after acylation to N-(4-fluoro-2-methylphenyl)acetamide of formula (VI) (CAS No. 326-65-8), to give 4-fluoro-2-methyl-5-nitroaniline of formula (VII) (CAS No. 446-18-4) or N-(4-fluoro-2-methyl-5-nitrophenyl)acetamide of formula (VIII) (CAS No. 273401-26-6). The 4-fluoro-2-methyl-5-nitroaniline can subsequently be converted in a manner known in principle by diazotization and reduction with hypophosphorous acid to 2-fluoro-4-methylnitrobenzene of formula (II) (see scheme 3). However, this route has many steps, has the disadvantage of an at most only 75% yield in the nitration to (IV), and should in addition be judged critically in terms of safety aspects since the 4-fluoro-2-methyl-5-nitroaniline of formula (VII) has a high energy content of more than 3300 J/g.

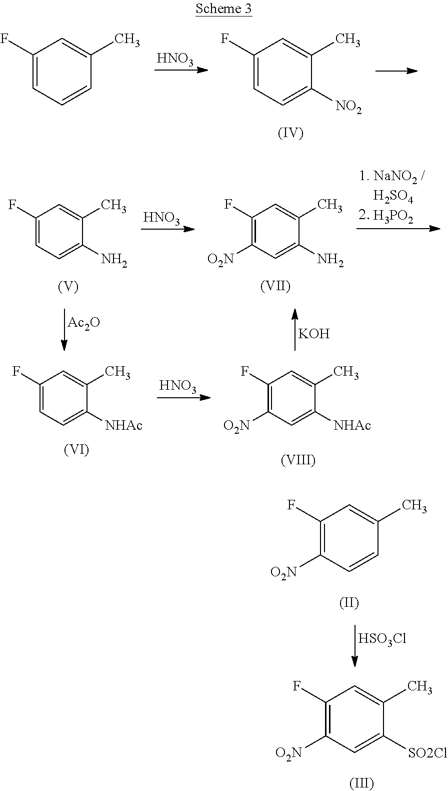

In principle it is furthermore also possible to obtain the sulfonyl chloride of formula (III) via 4-fluoro-2-methyl-5-nitroaniline of formula (VII) by means of a Meerwein reaction. However, this route (see scheme 4) is only one step shorter than the aforementioned route and still has the disadvantages mentioned for the synthesis by scheme 3.

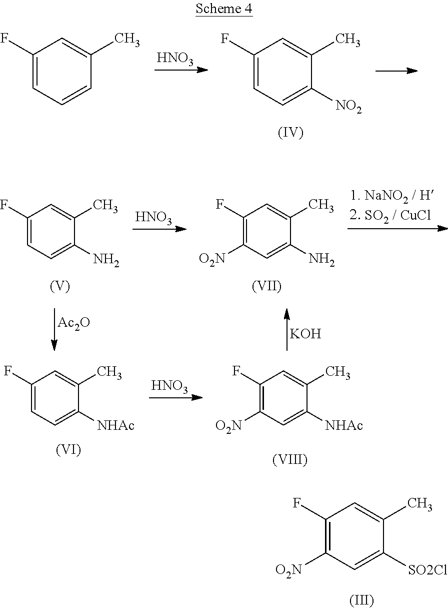

It has likewise already been disclosed (cf. for example WO 2000/66562) to subject 3-fluorotoluene first to a sulfochlorination to obtain the 4-fluoro-2-methylbenzenesulfonyl chloride of formula (IX) (CAS No. 7079-48-3) as main product. Subsequent nitration (cf. for example WO 2011/123609) leads to the 4-fluoro-2-methyl-5-nitrobenzenesulfonyl chloride of formula (III), which can be converted to the 1,1′-disulfanediylbis(4-fluoro-2-methyl-5-nitrobenzene) of formula (I) by methods known in principle (see scheme 5).

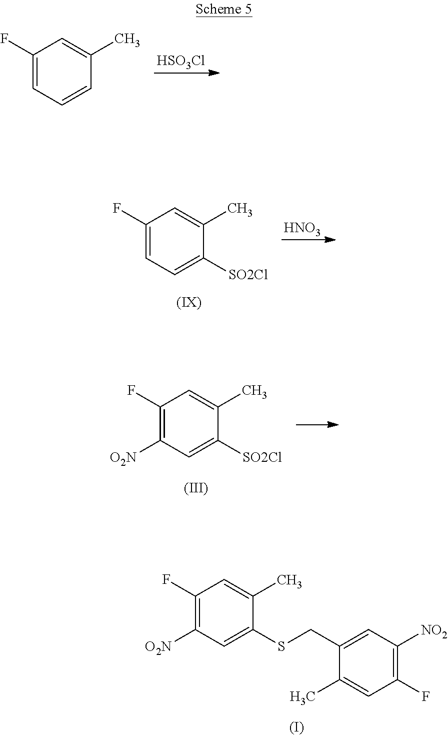

While the synthesis of the compound of formula (I) via the route corresponding to scheme 5 is short, and hence is the most economical of the syntheses known to date, it has the disadvantage that isomeric products are formed in each case both in the first step (sulfochlorination of 3-fluorotoluene) and in the second step (nitration). For instance, in the sulfochlorination of 3-fluorotoluene, in addition to the desired product 4-fluoro-2-methylbenzenesulfonyl chloride of formula (IX), 2-fluoro-4-methylbenzenesulfonyl chloride of formula (X) (CAS No 518070-29-6) is also obtained in proportions of around 10%; the third possible isomer, 2-fluoro-6-methylbenzenesulfonyl chloride (CAS No. 1092350-02-1) in this case only has an occurrence of around 1%. Although these sulfonyl chlorides can in principle be separated from one another by distillation, this is a time- and energy-consuming step which in addition has a price in terms of yield. In addition, in the next step of the synthesis (nitration), two isomeric nitrosulfonyl chlorides are formed even when using isomerically pure 4-fluoro-2-methylbenzenesulfonyl chloride of formula (IX): In addition to the desired 4-fluoro-2-methyl-5-nitrobenzenesulfonyl chloride of formula (III), the undesired 4-fluoro-2-methyl-3-nitrobenzenesulfonyl chloride of formula (XI) (CAS No. 1158963-96-8) is also formed.

There was therefore a continuing need for a simple, economically advantageous method for preparing 1,1′-disulfanediylbis(4-fluoro-2-methyl-5-nitrobenzene) of formula (I) which could be reliably performed on an industrial scale and overcame at least some of the disadvantages mentioned above.

This object has surprisingly now been achieved by a process according to Claim 1, which is characterized in that

in a first process step (1), 3-fluorotoluene is reacted with chlorosulfonic acid to give a first mixture comprising 4-fluoro-2-methylbenzenesulfonyl chloride of formula (IX) and 2-fluoro-4-methylbenzenesulfonyl chloride of formula (X),

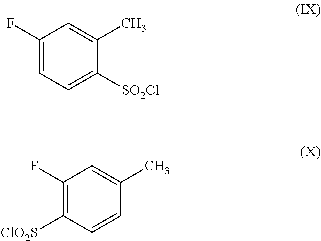

in a second process step (2), the first mixture from step (1) is nitrated with nitric acid to give a second mixture comprising 4-fluoro-2-methyl-5-nitrobenzenesulfonyl chloride of formula (III), 4-fluoro-2-methyl-3-nitrobenzenesulfonyl chloride of formula (XI) and 2-fluoro-4-methyl-5-nitrobenzenesulfonyl chloride of formula (XII),

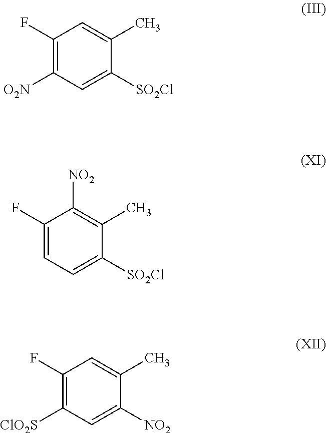

in a third process step (3), the second mixture from step (2) is converted to a third mixture by reducing the amount of 4-fluoro-2-methyl-3-nitrobenzenesulfonyl chloride of formula (XI) by at least 50%, based on the starting amount of 4-fluoro-2-methyl-3-benzenesulfonyl chloride of formula (XI) in the second mixture, and

in a fourth process step (4), the third mixture from step (3) is reduced to give a fourth mixture, comprising 1,1′-disulfanediylbis(4-fluoro-2-methyl-5-nitrobenzene) of formula (I) and at least one further compound (A) selected from

1,1′-disulfanediylbis(4-fluoro-2-methyl-3 -nitrobenzene) of formula (XIII),

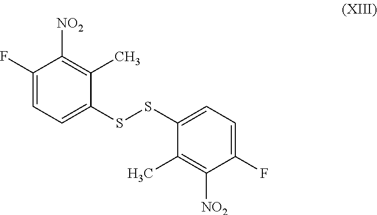 [SMILES: Cc1c(SSc2ccc(F)c([N+](=O)[O-])c2C)ccc(F)c1[N+](=O)[O-]]

1,1′-disulfanediylbis(2-fluoro-4-methyl-5-nitrobenzene) of formula (XIV),

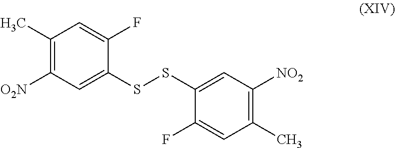 [SMILES: Cc1cc(F)c(SSc2cc([N+](=O)[O-])c(C)cc2F)cc1[N+](=O)[O-]]

1-fluoro-4-[(4-fluoro-2-methyl-5-nitrophenyl)disulfanyl]-3-methyl-2-nitrobenzene of formula (XV),

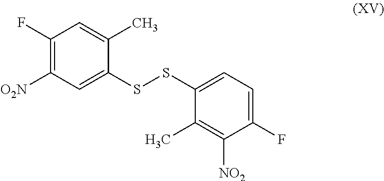 [SMILES: Cc1cc(F)c([N+](=O)[O-])cc1SSc1ccc(F)c([N+](=O)[O-])c1C]

1-fluoro-4-[(2-fluoro-4-methyl-5-nitrophenyl)disulfanyl]-5-methyl-2-nitrobenzene of formula (XVI),

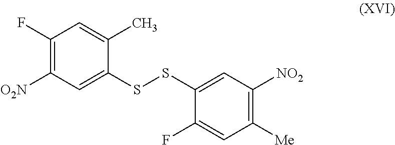 [SMILES: Cc1cc(F)c([N+](=O)[O-])cc1SSc1cc([N+](=O)[O-])c(C)cc1F]

and

1-fluoro-4-[(2-fluoro-4-methyl-5-nitrophenyl)disulfanyl]-3-methyl-2-nitrobenzene of formula (XVII).

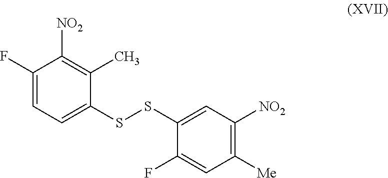 [SMILES: Cc1cc(F)c(SSc2ccc(F)c([N+](=O)[O-])c2C)cc1[N+](=O)[O-]]

Surprisingly, 1,1′-disulfanediylbis(4-fluoro-2-methyl-5-nitrobenzene) of formula (I) can be prepared with good yields and in high purity with a process according to the invention. It has been found that the sulfonyl chlorides of formulae (IX) and (X) can be converted together according to the invention, that is to say without any complex separation of the same, provided that the amount of compound (XI) also formed in the nitration is subsequently reduced before carrying out the reduction according to process step 4. Instead of having to work up the sulfonyl chlorides of formulae (IX) and (X), it is sufficient for a good yield and purity of the compound of formula (I) to instead reduce the amount of compound of formula (XI), which is simpler to implement in terms of process engineering, for example by crystallization. In addition, a process according to the invention allows solvents to be partly dispensed with since some process steps can be carried out without solvent. In this way the requirement for solvents can be reduced as a whole. In addition, a process according to the invention allows the use of solvents suitable for the industrial scale in the process steps for which a solvent is provided or helpful. A further advantage is that a process according to the invention makes it possible to obtain the desired target compounds without the need for complex and hence expensive purification methods between the individual synthesis steps. Reaction mixtures obtained from a process step can in some cases even be used in the next step of the process according to the invention without further purification and separation of the mixture, or the purification step provided can be carried out using a comparatively simple purification method. As a result, the process according to the invention is economically advantageous. In addition, it can also be reliably carried out on the industrial scale.

The process according to the invention is illustrated in Scheme 6.

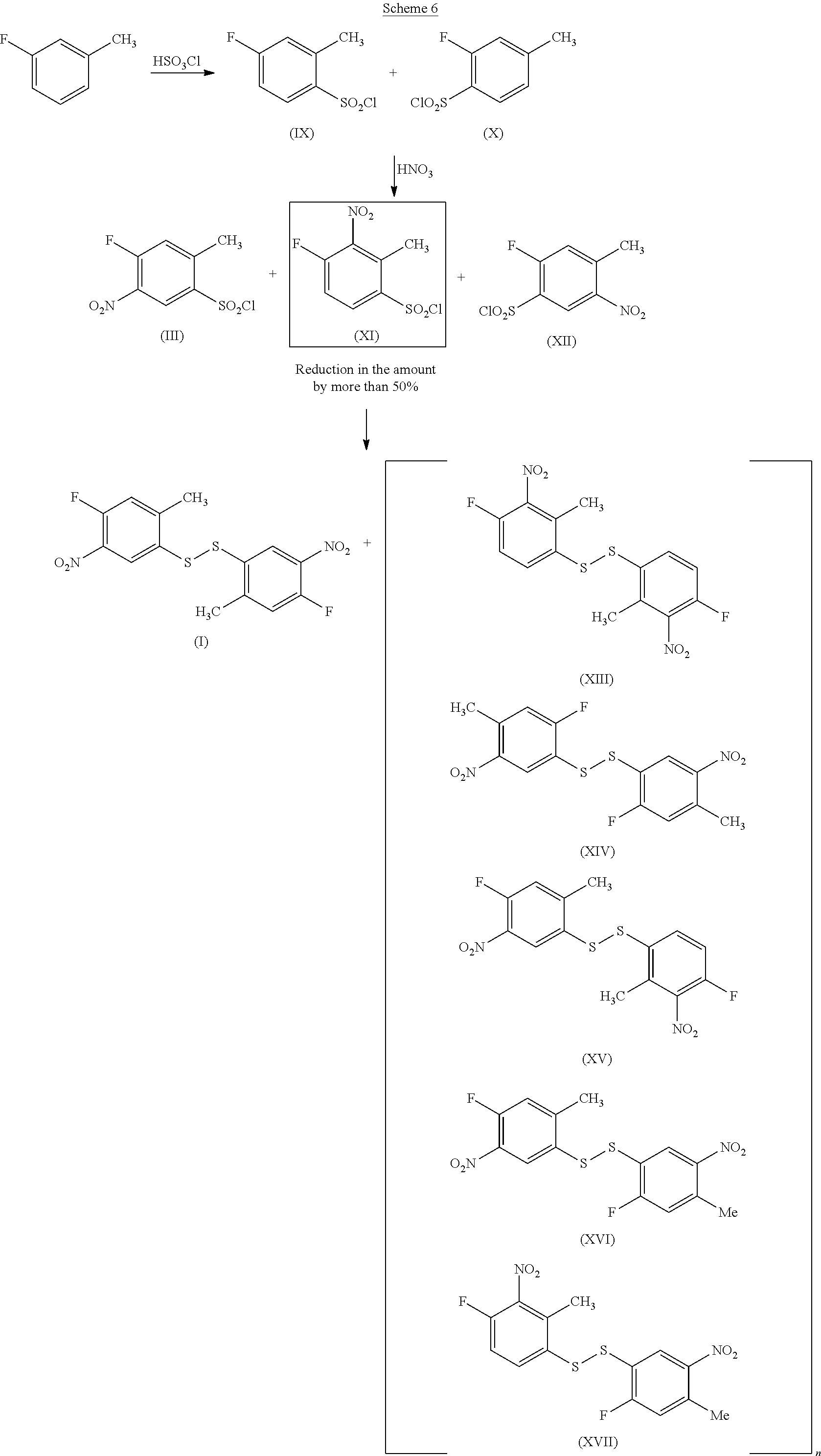

In the first step (1) of the process according to the invention, 3-fluorotoluene is reacted with chlorosulfonic acid to give a mixture of 4-fluoro-2-methylbenzenesulfonyl chloride of formula (IX) and 2-fluoro-4-methylbenzenesulfonyl chloride of formula (X).

In a preferred embodiment, this first step (1) of the process according to the invention is further characterized in that 3-fluorotoluene is chlorosulfonated without solvent in the presence of 2 to 5 molar equivalents of chlorosulfonic acid. Particular preference is given to using 2.5 to 4 molar equivalents of chlorosulfonic acid.

The reaction is preferably carried out at a temperature between −5 and 40° C. and particularly preferably between 0 and 25° C.

The reaction mixture from this first step (1) can then be used in the second step (2) of the process according to the invention without further purification and separation of the isomers.

In a further embodiment of the process according to the invention, the reaction mixture from the first step (1) is worked up, likewise preferably without addition of a solvent, by adding three to 30 kilograms of water per kilogram of 3-fluorotoluene, preferably four to 25 kilograms of water per kilogram of 3-fluorotoluene, and the phases are subsequently separated. The organic phase is then used in the second step (2) of the process according to the invention, preferably without further purification.

In the second step (2) of the process according to the invention, the mixture obtained after process step 1 (“first mixture”) and comprising the sulfonyl chlorides (IX) and (X) is nitrated. This nitration is preferably conducted in sulfuric acid as solvent.

The amount of sulfuric acid is between 1 and 20 molar equivalents, based on the mixture of the sulfonyl chlorides (IX) and (X). Preference is given to using between 1 and 10 molar equivalents, based on the mixture of the sulfonyl chlorides (IX) and (X).

According to the invention, the nitration is conducted using nitric acid, preferably using 70 to 100% nitric acid. Particular preference is given to using 90 to 100% nitric acid.

The amount of nitric acid is between 1 and 1.75 molar equivalents, based on the mixture of the sulfonyl chlorides (IX) and (X). Preference is given to using between 1.2 and 1.5 molar equivalents, based on the mixture of the sulfonyl chlorides (IX) and (X).

The reaction is preferably carried out at a temperature between −5 and 70° C. and particularly preferably between 0 and 40° C.

This second process step results in the obtaining of a reaction mixture (“second mixture”) comprising 4-fluoro-2-methyl-5-nitrobenzenesulfonyl chloride of formula (III), 4-fluoro-2-methyl-3-nitrobenzenesulfonyl chloride of formula (XI) and 2-fluoro-4-methyl-5-nitrobenzenesulfonyl chloride of formula (XII).

In an optional embodiment of the process according to the invention, the second process step further comprises the workup of the resulting reaction mixture, preferably by crystallization (step (2a)). Here, the reaction mixture is firstly seeded with 4-fluoro-2-methyl-5-nitrobenzenesulfonyl chloride (III) and subsequently admixed with water, with between 1 and 10 kilograms of water (preferably also a mixture of water and ice) per kilogram of crude product (second mixture), and preferably between 4 and 5 kilograms of water (preferably also a mixture of water and ice) per kilogram of crude product (second mixture), typically being used. The resulting product is then isolated by filtration and washed with water, and then represents the result of this second process step, that is to say the obtained second mixture. The filtration and the subsequent washing of the crude product is performed by methods which are known in principle and familiar to those skilled in the art. For example, washing is performed once or twice with each time 1 to 3 kilograms of water per kilogram of filtration product or 1.5 to 2 kilograms of water per kilogram of filtration product. The amount of water can typically be varied depending on requirements.

As an optional alternative to step (2a), instead of the crystallization it is also possible to extract the reaction mixture formed in process step (2) with the solvent (step (2b)), provided that the solvent is suitable for this. A solvent is preferably suitable in this context if it cannot be nitrated and is acid stable. Accordingly, the product then obtained represents the result of this second process step, that is to say the obtained second mixture.

In the third step (3) of the process according to the invention, the mixture obtained after process step 2 (“second mixture”) and comprising the nitrosulfonyl chlorides (III), (XI) and (XII) is purified by reducing the amount of 4-fluoro-2-methyl-3-nitrobenzenesulfonyl chloride of formula (XI) by at least 50%, based on the starting amount thereof in the second mixture.

It is desirable and accordingly preferred for the 4-fluoro-2-methyl-3-nitrobenzenesulfonyl chloride of formula (XI) to be largely removed. The amount of 4-fluoro-2-methyl-3-nitrobenzenesulfonyl chloride of formula (XI) is therefore reduced, with increasing preference, by at least 60%, at least 70%, at least 80%, at least 85%, at least 90%, at least 93% and very particularly preferably by at least 95%.

The purification, that is to say the reduction in the amount of 4-fluoro-2-methyl-3-nitrobenzenesulfonyl chloride of formula (XI), is preferably effected by crystallization. The crystallization is conducted in a solvent. Solvents used for the crystallization in this process step are toluene, o-xylene, m-xylene, p-xylene, mesitylene, chlorobenzene, pentane, hexane, heptane, octane, isooctane, cyclopentane, cyclohexane, methylcyclohexane, decalin, special boiling point spirit 60/95, special boiling point spirit 80/110, special boiling point spirit 80/120, special boiling point spirit 100/125, special boiling point spirit 100/140, special boiling point spirit 100/155 or mixtures of these solvents. Preference is given to using heptane, octane, isooctane, methylcyclohexane, special boiling point spirit 100/125, special boiling point spirit 100/140, special boiling point spirit 100/155 or mixtures of these solvents. More preference is given to using heptane, octane, isooctane, methylcyclohexane, special boiling point spirit 100/125, special boiling point spirit 100/140, special boiling point spirit 100/155 or mixtures of these solvents. Particular preference is given to using heptane, isooctane, methylcyclohexane, special boiling point spirit 100/155 or mixtures of these solvents.

The amount of solvent used for the crystallization is between 1 and 10 kilograms per kilogram of crude product (second mixture), preferably between 1 and 5 kilograms per kilogram of crude product (second mixture).

The crystallization is conducted at a temperature between −10 and 30° C., preferably between 0 and 25° C.

This third process step results in the obtaining of a reaction mixture (“third mixture”) in which the amount of 4-fluoro-2-methyl-3-nitrobenzenesulfonyl chloride of formula (XI) has been accordingly reduced as described hereinabove.

In the fourth step (4) of the process according to the invention, the mixture of nitrosulfonyl chlorides obtained after process step 3 (“third mixture”) is reduced. This results in a fourth mixture comprising the desired 1,1′-disulfanediylbis(4-fluoro-2-methyl-5-nitrobenzene) of formula (I) and at least one further compound (A) selected from 1,1′-disulfanediylbis(4-fluoro-2-methyl-3 -nitrobenzene) of formula (XIII) (CAS No. 1613615-92-1), 1,1′-disulfanediylbis(2-fluoro-4-methyl-5-nitrobenzene) of formula (XIV) (CAS No. 1613615-90-9), 1-fluoro-4-[(4-fluoro-2-methyl-5 -nitrophenyl)disulfanyl]-3-methyl-2-nitrobenzene of formula (XV) (CAS No. 1613615-95-4), 1-fluoro-4-[(2-fluoro-4-methyl-5-nitrophenyl)disulfanyl]-5-methyl-2-nitrobenzene of formula (XVI) (CAS No. 1613615-93-2), and 1-fluoro-4-[(2-fluoro-4-methyl-5-nitrophenyl)disulfanyl]-3-methyl-2-nitrobenzene of formula (XVII). The one or more compounds (A) are isomeric reaction products. In the fourth process step according to the invention, in addition to the desired compound of formula (I), one, two, three, four or all five of compounds (A) are formed.

The reduction in the fourth step of the process according to the invention can be carried out by methods known in principle. By way of example, it can be conducted using sodium hypophosphite, sodium hypophosphite hydrate or ascorbic acid, which is preferred, and preferably in the presence of a catalyst, in particular a catalytic amount of iodide. The reduction is particularly preferably conducted using sodium hypophosphite and a catalytic amount of iodide, for example potassium iodide. The amount of sodium hypophosphite used for the reduction is for example between 0.25 and 2 kilograms per kilogram of crude product (third mixture), preferably between 0.5 and 1 kilogram per kilogram of crude product (third mixture). The amount of ascorbic acid used for the reduction is for example between 0.25 and 2 kilograms per kilogram of crude product (third mixture), preferably between 0.5 and 1 kilogram per kilogram of crude product (third mixture).

Alternative reducing agents and/or catalysts are known to those skilled in the art and are likewise contemplated.

Solvents useful for the fourth step of the process according to the invention are formic acid, acetic acid, propionic acid or mixtures of these solvents. Preference is given to using acetic acid. The amount of solvent used is between 1 and 10 kilograms per kilogram of crude product (third mixture), preferably between 2 and 7 kilograms per kilogram of crude product (third mixture) and more preferably between 3 and 4 kilograms per kilogram of crude product (third mixture). The amount of solvent in this process step can typically be varied depending on requirements.

This fourth process step results in the obtaining of a reaction mixture (“fourth mixture”) comprising 1,1′-disulfanediylbis(4-fluoro-2-methyl-5-nitrobenzene) of formula (I) and at least one further compound (A) as described hereinabove. Of course, this fourth mixture may also contain a plurality of or all of the compounds (A) as specified above.

The process according to the invention can be supplemented by a fifth process step in order to further increase the amount of the compound of formula (I) in the mixture formed.

A further embodiment of the process according to the invention is accordingly characterized in that in a fifth process step (5) the fourth mixture from process step (4) is converted to a fifth mixture by reducing the amount of one or more compounds (A) each by at least 50%, and with increasing preference each by at least 60%, each by at least 70%, each by at least 80%, each by at least 85%, each by at least 90%, each by at least 93% and very particularly preferably each by at least 95%, based on the starting amount of each compound (A) in the fourth mixture. The fourth mixture from process step (4) is thus purified by one or more compounds (A), that is to say one or more of the isomeric reaction products, being removed from the fourth mixture as comprehensively as possible.

In a further embodiment of the process according to the invention, the amount of two or more compounds (A) is reduced accordingly (i.e. as indicated above).

In a further embodiment of the process according to the invention, the amount of three or more compounds (A) is reduced accordingly (i.e. as indicated above).

In a further, preferred embodiment of the process according to the invention, at least one compound (A) is a compound of formula (XV) or a compound of formula (XVI). It is naturally likewise possible and preferred for both compounds (XV) and (XVI) mentioned to be present in the fourth mixture to be purified and consequently for both compounds to be present as a respective compound (A).

The purification, that is to say the reduction in the amount of one or more compounds (A), is preferably effected by crystallization. The crystallization is conducted in a solvent. The solvents used for the crystallization in this process step are formic acid, acetic acid, propionic acid or mixtures of these solvents. Preference is given to using acetic acid.

The amount of solvent for the crystallization is between 1 and 5 kilograms per kilogram of crude product (fourth mixture). Preference is given to using between 1 and 2 kilograms per kilogram of crude product (fourth mixture).

The crystallization is conducted at a temperature between 0 and 100° C., preferably between 10 and 50° C.

The process according to the invention shall be illustrated by the examples which follow, without being limited thereto.

### EXAMPLE 1

### 4-Fluoro-2-methylbenzenesulfonyl chloride (IX)

90 g (0.749 mol) of chlorosulfonic acid (97% purity) was initially charged and cooled to from 0 to 5° C. 27.8 g (0.25 mol) of 3-fluorotoluene (99% purity) were metered in at this temperature within 80 minutes. The mixture was subsequently stirred for a further 4 hours at 0 to 5° C., left overnight to come to room temperature and then the reaction mixture was stirred into 700 g of ice-water, the temperature not rising above 10° C. The emulsion obtained was then extracted three times with 100 ml of methylene chloride each time. The combined organic phases were concentrated under a gentle vacuum. This gave 44.4 g of a yellowish oil.

Composition:

HPLC: 86.1 area % 4-fluoro-2-methylbenzenesulfonyl chloride (IX) (corresponding to 73% of theory)
8.1 area % 2-fluoro-4-methylbenzenesulfonyl chloride (X)

### EXAMPLE 2

### 4-Fluoro-2-methylbenzenesulfonyl chloride (IX)

72.1 g (0.6 mol) of chlorosulfonic acid (97% purity) was initially charged and cooled to from 0 to 5° C. 22.25 g (0.2 mol) of 3-fluorotoluene (99% purity) were metered in at this temperature within 120 minutes. The mixture was subsequently stirred for a further 2 hours at 10 to 12° C. The reaction mixture was subsequently metered at 45 to 50° C. into 100 g of water and the phases were separated. This gave 33.3 g of a cloudy oil.

Composition:

HPLC: 87.3 area % 4-fluoro-2-methylbenzenesulfonyl chloride (IX) (corresponding to 70% of theory)
9.0 area % 2-fluoro-4-methylbenzenesulfonyl chloride (X)

### EXAMPLE 3

### 4-Fluoro-2-methyl-5-nitrobenzenesulfonyl chloride (III)

35.7 ml of conc. sulfuric acid (65.38 g; 12.8 molar equivalents, based on the sum total of the isomeric fluoromethylbenzenesulfonyl chlorides) were initially charged and to this were metered in at an internal temperature of 5° C. 10.61 g (0.05 mol) of 4-fluoro-2-methylbenzenesulfonyl chloride having a purity of 90% (additionally containing 8.3% of 2-fluoro-4-methylbenzenesulfonyl chloride). Then, at an internal temperature of 0 to 5° C., 5.85 g (0.065 mol) of 70% nitric acid (1.3 molar equivalents, based on the sum total of the isomeric fluoromethylbenzenesulfonyl chlorides) were metered in within 10 minutes. After the end of the metered addition of nitric acid, the mixture was stirred further for 1 hour at 10 to 15° C. The reaction mixture (suspension) was then stirred into 200 ml of ice-water. Extraction was performed twice with 50 ml of methylene chloride, the combined organic phases were washed once with 30 ml of water, dried and concentrated under reduced pressure. This gave 12.7 g of a yellowish oil which solidified in crystalline form after some time.

Composition:

HPLC: 87.5 area % sum total of 4-fluoro-2-methyl-5-nitrobenzenesulfonyl chloride (III) and 2-fluoro-4-methyl-5-nitrobenzenesulfonyl chloride (XII)
9.2 area % 4-fluoro-2-methyl-3-nitrobenzenesulfonyl chloride (XI)

¹⁹F NMR: 83.0% 4-fluoro-2-methyl-5-nitrobenzenesulfonyl chloride (III) (−106.2 ppm)
8.6% 2-fluoro-4-methyl-5-nitrobenzenesulfonyl chloride (XII) (−100.2 ppm)
8.3% 4-fluoro-2-methyl-3-nitrobenzenesulfonyl chloride (XI) (−111.3 ppm)

### EXAMPLE 4

### 4-Fluoro-2-methyl-5-nitrobenzenesulfonyl chloride (III)

22.3 ml of conc. sulfuric acid (40.87 g; 4 molar equivalents, based on the sum total of the isomeric fluoromethylbenzenesulfonyl chlorides) were initially charged and to this were metered in at an internal temperature of 20° C. 21.22 g (0.1 mol) of 4-fluoro-2-methylbenzenesulfonyl chloride having a purity of 90% (additionally containing 8.3% of 2-fluoro-4-methylbenzenesulfonyl chloride). Then, at an internal temperature of 20 to 23° C., 7.88 g (0.125 mol) of 100% nitric acid (1.25 molar equivalents, based on the sum total of the isomeric fluoromethylbenzenesulfonyl chlorides) were metered in within 15 minutes. After the end of the metered addition of nitric acid, the mixture was stirred further for 2 hours at 20 to 22° C. The reaction mixture (suspension) was then stirred into 300 ml of ice-water. Extraction was performed twice with 50 ml of methylene chloride, the combined organic phases were washed once with 30 ml of water, dried and concentrated under reduced pressure. This gave 25.7 g of a yellowish oil which solidified in crystalline form after some time.

Composition:

HPLC: 86.3 area % sum total of 4-fluoro-2-methyl-5-nitrobenzenesulfonyl chloride (III) and 2-fluoro-4-methyl-5-nitrobenzenesulfonyl chloride (XII)
10.6 area % 4-fluoro-2-methyl-3-nitrobenzenesulfonyl chloride (XI)

¹⁹F NMR: 81.9% 4-fluoro-2-methyl-5-nitrobenzenesulfonyl chloride (III) (−106.2 ppm)
8.7% 2-fluoro-4-methyl-5-nitrobenzenesulfonyl chloride (XII) (−100.2 ppm)
9.4% 4-fluoro-2-methyl-3-nitrobenzenesulfonyl chloride (XI) (−111.3 ppm)

### EXAMPLE 5

### 4-Fluoro-2-methyl-5-nitrobenzenesulfonyl chloride (III)

51 ml of conc. sulfuric acid (57.2 g; 4 molar equivalents, based on the sum total of the isomeric fluoromethylbenzenesulfonyl chlorides) were initially charged and to this were metered in at an internal temperature of 20 to 25° C. 30.33 g (0.14 mol) of a mixture having the composition 90.0% 4-fluoro-2-methylbenzenesulfonyl chloride and 6.3% 2-fluoro-4-methylbenzenesulfonyl chloride. Then, at an internal temperature of 20 to 25° C., 11.03 g (0.175 mol) of 100% nitric acid (1.25 molar equivalents, based on the sum total of the isomeric fluoromethylbenzenesulfonyl chlorides) were metered in within 190 minutes. After the end of the metered addition of nitric acid, the mixture was stirred further for 2 hours at 30 to 35° C. The phases were then separated at this temperature. The upper phase was stirred into 140 ml of water. The precipitated solids were filtered off, washed with water and dried under reduced pressure at 40° C. This gave 31.84 g of a yellowish solid.

Composition:

HPLC: 84.9 area % sum total of 4-fluoro-2-methyl-5-nitrobenzenesulfonyl chloride (III) and 2-fluoro-4-methyl-5-nitrobenzenesulfonyl chloride (XII)
11.0 area % 4-fluoro-2-methyl-3-nitrobenzenesulfonyl chloride (XI)

¹⁹F NMR: 81.9% 4-fluoro-2-methyl-5-nitrobenzenesulfonyl chloride (III) (−106.2 ppm)
8.0% 2-fluoro-4-methyl-5-nitrobenzenesulfonyl chloride (XII) (−100.2 ppm)
10.2% 4-fluoro-2-methyl-3-nitrobenzenesulfonyl chloride (XI) (−111.3 ppm)

31.05 g of this solid were stirred into 100 ml of methylcyclohexane (MCH) for 1 hour at room temperature.

The remaining solid was then filtered off with suction, washed with 20 ml of MCH and dried under reduced pressure at 45° C. This gave 25.77 g of colourless solid.

Composition:

HPLC: 94.8 area % sum total of 4-fluoro-2-methyl-5-nitrobenzenesulfonyl chloride (III) and 2-fluoro-4-methyl-5-nitrobenzenesulfonyl chloride (XII)
1.8 area % 4-fluoro-2-methyl-3-nitrobenzenesulfonyl chloride (XI)

¹⁹F NMR: 91.0% 4-fluoro-2-methyl-5-nitrobenzenesulfonyl chloride (III) (−106.2 ppm)
6.5% 2-fluoro-4-methyl-5-nitrobenzenesulfonyl chloride (XII) (−100.2 ppm)
1.7% 4-fluoro-2-methyl-3-nitrobenzenesulfonyl chloride (XI) (−111.3 ppm)

### EXAMPLE 6

### 4-Fluoro-2-methyl-5-nitrobenzenesulfonyl chloride (III)

A reaction vessel was initially charged with 418.7 ml (766.2 g; 3 molar equivalents) of conc. sulfuric acid and this was cooled to 10° C. Subsequently, first 330 g (1.525 mol) of a mixture having the composition 92.4% 4-fluoro-2-methylbenzenesulfonyl chloride and 4% 2-fluoro-4-methylbenzenesulfonyl chloride and 211 g (0.979 mol) of a mixture having the composition 91.1% 4-fluoro-2-methylbenzenesulfonyl chloride and 5.7% 2-fluoro-4-methylbenzenesulfonyl chloride were metered in and thereafter 196.9 g (3.125 mol) of 100% nitric acid (1.25 molar equivalents, based on the sum total of the isomeric fluoromethylbenzenesulfonyl chlorides) were metered in within 120 minutes. After the end of the metered addition of nitric acid, the mixture was stirred further for 7 days at 20 to 25° C. The reaction mixture was then dissolved in 800 ml of methylene chloride. The solution was stirred into 1000 ml of ice-water, the phases were separated, the aqueous phase was extracted with 200 ml of methylene chloride and the combined organic phases were washed twice with 750 ml of water each time. After drying over sodium sulfate and concentrating under reduced pressure, this gave 644.8 g of a yellowish oil which later solidified.

Composition:

HPLC: 85.0 area % sum total of 4-fluoro-2-methyl-5-nitrobenzenesulfonyl chloride (III) and 2-fluoro-4-methyl-5-nitrobenzenesulfonyl chloride (XII)
10.4 area % 4-fluoro-2-methyl-3-nitrobenzenesulfonyl chloride (XI)

¹⁹F NMR: 83.1% 4-fluoro-2-methyl-5-nitrobenzenesulfonyl chloride (III) (−106.2 ppm)
6.5% 2-fluoro-4-methyl-5-nitrobenzenesulfonyl chloride (XII) (−100.2 ppm)
8.5% 4-fluoro-2-methyl-3-nitrobenzenesulfonyl chloride (XI) (−111.3 ppm)

25 g of the product thus prepared were initially charged in 115.5 g of methylcyclohexane and the mixture was heated with stirring to 83° C., until a clear solution was formed. This solution was then slowly cooled to 20° C. and stirred at this temperature for a further 3 hours. The solid was filtered off with suction, washed with a little MCH and dried. This gave 19.2 g of a pale yellow solid.

Composition:

HPLC: 94.0 area % sum total of 4-fluoro-2-methyl-5-nitrobenzenesulfonyl chloride (III) and 2-fluoro-4-methyl-5-nitrobenzenesulfonyl chloride (XII)
1.1 area % 4-fluoro-2-methyl-3-nitrobenzenesulfonyl chloride (XI)

¹⁹F NMR: 92.2% 4-fluoro-2-methyl-5-nitrobenzenesulfonyl chloride (III) (−106.2 ppm)
6.9% 2-fluoro-4-methyl-5-nitrobenzenesulfonyl chloride (XII) (−100.2 ppm)
<0.1% 4-fluoro-2-methyl-3-nitrobenzenesulfonyl chloride (XI) (−111.3 ppm)

### EXAMPLE 7

### 1,1′-disulfanediylbis(4-fluoro-2-methyl-5-nitrobenzene) (I)

### a) Synthesis

63.0 g of crude (not purified by crystallization from MCH) of 4-fluoro-2-methyl-5-nitrobenzenesulfonyl chloride having the composition according to HPLC of 84.3 area % sum total of 4-fluoro-2-methyl-5-nitrobenzenesulfonyl chloride (III) and 2-fluoro-4-methyl-5-nitrobenzenesulfonyl chloride (XII) and 10.8 area % 4-fluoro-2-methyl-3-nitrobenzenesulfonyl chloride (XI) (approx. 95.1%, sum total of the isomers, corresponding to 0.236 mol) were initially charged in 250 g of acetic acid, 3.92 g (23.6 mmol) of potassium iodide were added and then 37.56 g (0.354 mol) of sodium hypophosphite monohydrate were metered in at 60° C. within 100 minutes. The mixture was stirred at 60° C. for 5 hours, then cooled to 40° C., 100 ml of water were added, the mixture was stirred at 30° C. for 30 minutes, and the solid was filtered off, washed with 60 ml of water and dried. This gave 41.65 g of solid.

Composition:

HPLC: 64.2 area % 1,1′-disulfanediylbis(4-fluoro-2-methyl-5-nitrobenzene) (I)
13.3 area % 1-fluoro-4-[(2-fluoro-4-methyl-5-nitrophenyl)disulfanyl]-5-methyl-2-nitrobenzene (XVI)
18.1 area % 1-fluoro-4-[(4-fluoro-2-methyl-5-nitrophenyl)disulfanyl]-3-methyl-2-nitrobenzene (XV)
1.8 area % 1-fluoro-4-[(2-fluoro-4-methyl-5-nitrophenyl)disulfanyl]-3-methyl-2-nitrobenzene (XVII)
1.2 area % 1,1′-disulfanediylbis(4-fluoro-2-methyl-3-nitrobenzene) (XIII)

### b) Purification

The solid from a) was dissolved in 84 g of acetic acid at approx. 80° C. The solution was left to cool to 20° C. with stirring, and the precipitated crystals were filtered off, washed with a little petroleum ether and dried. 32.5 g of solid were obtained.

Composition:

HPLC: 79.7 area % 1,1′-disulfanediylbis(4-fluoro-2-methyl-5-nitrobenzene) (I)
4.3 area % 1-fluoro-4-[(2-fluoro-4-methyl-5-nitrophenyl)disulfanyl]-5-methyl-2-nitrobenzene (XVI)
14.8 area % 1-fluoro-4-[(4-fluoro-2-methyl-5-nitrophenyl)disulfanyl]-3-methyl-2-nitrobenzene (XV)
0.4 area % 1-fluoro-4-[(2-fluoro-4-methyl-5-nitrophenyl)disulfanyl]-3-methyl-2-nitrobenzene (XVII)
0.7 area % 1,1′-disulfanediylbis(4-fluoro-2-methyl-3-nitrobenzene) (XIII)

### EXAMPLE 8

### 1,1′-disulfanediylbis(4-fluoro-2-methyl-5-nitrobenzene) (I)

### a) Synthesis

4.1 kg of 4-fluoro-2-methyl-5-nitrobenzenesulfonyl chloride having the composition according to HPLC of 93.3 area % sum total of 4-fluoro-2-methyl-5-nitrobenzenesulfonyl chloride (III) and 2-fluoro-4-methyl-5-nitrobenzenesulfonyl chloride (XII) and 2.3 area % 4-fluoro-2-methyl-3-nitrobenzenesulfonyl chloride (XI) (approx. 95.6%, sum total of isomers, corresponding to 15.45 mol) were initially charged in 30.5 kg of acetic acid, 0.25 kg (1.51 mol) of potassium iodide were added and then 2.388 kg (27.1 mol) of sodium hypophosphite were metered in at 40° C. within 100 minutes. The mixture was stirred at 40° C. for 16 hours, approx. 20 1 of acetic acid were removed by distillation, the residue was metered into 20 1 of water and this mixture was stirred at 40° C. for 1 hour. The precipitated solid was filtered off and washed with a total of 30 1 of water. After drying this gave 2.86 kg of yellow solid.

Composition:

HPLC: 81.8 area % 1,1′-disulfanediylbis(4-fluoro-2-methyl-5-nitrobenzene) (I)
10.2 area % 1-fluoro-4-[(2-fluoro-4-methyl-5-nitrophenyl)disulfanyl]-5-methyl-2-nitrobenzene (XVI)
3.4 area % 1-fluoro-4-[(4-fluoro-2-methyl-5-nitrophenyl)disulfanyl]-3-methyl-2-nitrobenzene (XV)
1.6 area % 1,1′-disulfanediylbis(2-fluoro-4-methyl-5-nitrobenzene) (XIV)

### b) Purification

4.4 kg of a solid which had been prepared analogously to example 8a) and had the following composition:

HPLC: 80.4 area % 1,1′-disulfanediylbis(4-fluoro-2-methyl-5-nitrobenzene) (I)
13.1 area % 1-fluoro-4-[(2-fluoro-4-methyl-5-nitrophenyl)disulfanyl]-5-methyl-2-nitrobenzene (XVI)
4.6 area % 1-fluoro-4-[(4-fluoro-2-methyl-5-nitrophenyl)disulfanyl]-3-methyl-2-nitrobenzene (XV)
0.3 area % 1,1′-disulfanediylbis(2-fluoro-4-methyl-5-nitrobenzene) (XIV)

were dissolved in 8.5 1 of acetic acid at 100° C. This solution was slowly cooled to 30° C., with crystallization commencing from approx. 60° C. The precipitated solid was filtered off with suction, washed with acetic acid and water and dried. This gave 3.52 kg of solid.

Composition: (sum total 97%)

HPLC: 96.9 area % 1,1′-disulfanediylbis(4-fluoro-2-methyl-5-nitrobenzene) (I)
2.7 area % 1-fluoro-4-[(2-fluoro-4-methyl-5-nitrophenyl)disulfanyl]-5-methyl-2-nitrobenzene (XVI)
0.3 area % 1-fluoro-4-[(4-fluoro-2-methyl-5-nitrophenyl)disulfanyl]-3-methyl-2-nitrobenzene (XV)
<0.1 area % 1,1′-disulfanediylbis(2-fluoro-4-methyl-5-nitrobenzene) (XIV)

It is clear that the amount of compound of formula (XI), which for example is reduced compared to example 7, brings about a better purity of the compound of formula (I).

1. A process for preparing 1,1′-disulfanediylbis(4-fluoro-2-methyl-5-nitrobenzene) of formula (I) 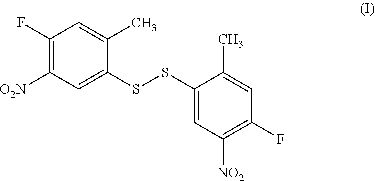 [SMILES: Cc1cc(F)c([N+](=O)[O-])cc1SSc1cc([N+](=O)[O-])c(F)cc1C]
comprising
(1) reacting, 3-fluorotoluene with chlorosulfonic acid to give a first mixture comprising 4-fluoro-2-methylbenzenesulfonyl chloride of formula (IX) and 2-fluoro-4-methylbenzenesulfonyl chloride of formula (X), 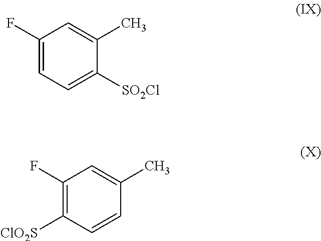
(2) nitrating, the first mixture from (1) with nitric acid to give a second mixture comprising 4-fluoro-2-methyl-5-nitrobenzenesulfonyl chloride of formula (III), 4-fluoro-2-methyl-3-nitrobenzenesulfonyl chloride of formula (XI) and 2-fluoro-4-methyl-5-nitrobenzenesulfonyl chloride of formula (XII), 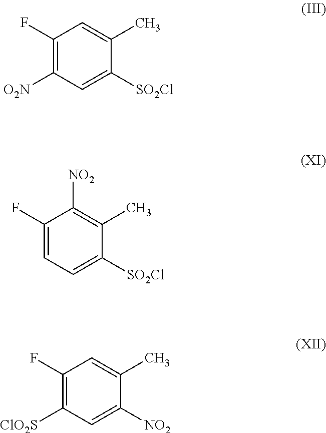
(3), converting the second mixture from (2) to a third mixture by reducing the amount of 4-fluoro-2-methyl-3-nitrobenzenesulfonyl chloride of formula (XI) by at least 50%, based on the starting amount of 4-fluoro-2-methyl-3-benzenesulfonyl chloride of formula (XI) in the second mixture,
(4), reducing the third mixture from (3) to give a fourth mixture, comprising 1,1′-disulfanediylbis(4-fluoro-2-methyl-5-nitrobenzene) of formula (I) and at least one further compound (A) selected from
1,1′-disulfanediylbis(4-fluoro-2-methyl-3-nitrobenzene) of formula (XIII), 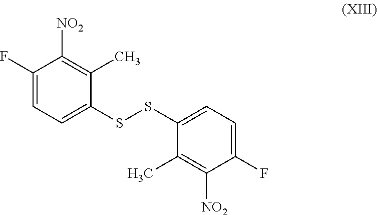 [SMILES: Cc1c(SSc2ccc(F)c([N+](=O)[O-])c2C)ccc(F)c1[N+](=O)[O-]]
1,1′-disulfanediylbis(2-fluoro-4-methyl-5-nitrobenzene) of formula (XIV), 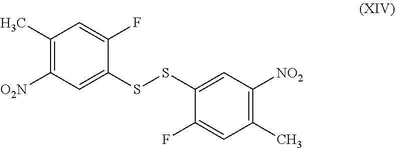 [SMILES: Cc1cc(F)c(SSc2cc([N+](=O)[O-])c(C)cc2F)cc1[N+](=O)[O-]]
1-fluoro-4-[(4-fluoro-2-methyl-5-nitrophenyl)disulfanyl]-3-methyl-2-nitrobenzene of formula (XV), 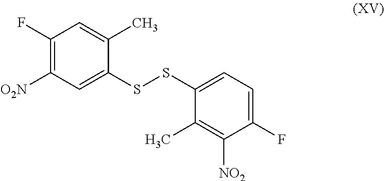 [SMILES: Cc1cc(F)c([N+](=O)[O-])cc1SSc1ccc(F)c([N+](=O)[O-])c1C]
1 -fluoro-4-[(2-fluoro-4-methyl-5-nitrophenyl)disulfanyl]-5-methyl-2-nitrobenzene of formula (XVI), 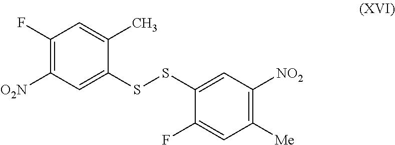 [SMILES: Cc1cc(F)c([N+](=O)[O-])cc1SSc1cc([N+](=O)[O-])c(C)cc1F]
and
1 -fluoro-4-[(2-fluoro-4-methyl-5-nitrophenyl)disulfanyl]-3-methyl-2-nitrobenzene of formula (XVII).

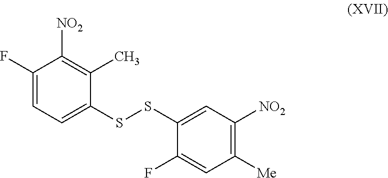 [SMILES: Cc1cc(F)c(SSc2ccc(F)c([N+](=O)[O-])c2C)cc1[N+](=O)[O-]]

2. The process according to claim 1, wherein
(1) 3-fluorotoluene is chlorosulfonated without solvent in the presence of 2 to 5 molar equivalents of chlorosulfonic acid.

3. The process according to claim 1, wherein (1) is carried out at a temperature between −5 and 40° C.

4. The process according to claim 1, wherein three to 30 kilograms of water per kilogram of 3-fluorotoluene are added to the first mixture after process step (1), optionally without addition of a solvent, phases are separated and an organic phase is used in (2).

5. The process according to claim 1, wherein (2) is conducted in sulfuric acid as solvent.

6. The process according to claim 5, wherein sulfuric acid is present between 1 and 20 molar equivalents, based on a mixture of sulfonyl chlorides of formulae (IX) and (X).

7. The process according to claim 1, wherein in (2) 70 to 100% nitric acid is used.

8. The process according to claim 7, wherein nitric acid is present between 1 and 1.75 molar equivalents, based on the mixture of sulfonyl chlorides of formulae (IX) and (X).

9. The process according to claim 1, wherein (2) is carried out at a temperature between −5 and 70° C.

10. The process according to claim 1, wherein the second mixture after (2) is additionally
a) seeded with 4-fluoro-2-methyl-5-nitrobenzenesulfonyl chloride of formula (III),
b) admixed with water,
c) filtered, and
d) washed with water.

11. The process according to claim 1, wherein the amount of 4-fluoro-2-methyl-3-nitrobenzenesulfonyl chloride of formula (XI) is reduced in (3) by crystallization in a solvent.

12. The process according to claim 11, wherein the solvent is toluene, o-xylene, m-xylene, p-xylene, mesitylene, chlorobenzene, pentane, hexane, heptane, octane, isooctane, cyclopentane, cyclohexane, methylcyclohexane, decalin, special boiling point spirit 60/95, special boiling point spirit 80/110, special boiling point spirit 80/120, special boiling point spirit 100/125, special boiling point spirit 100/140, special boiling point spirit 100/155 or a mixture thereof.

13. The process according to claim 11, wherein the amount of solvent is between 1 and 10 kilograms per kilogram of second mixture.

14. The process according to claim 11, wherein crystallization is conducted at a temperature between −10 and 30° C.

15. The process according to claim 1, wherein in (4) sodium hypophosphite, sodium hypophosphite hydrate or ascorbic acid is used as reducing agent.

16. The process according to claim 1, wherein (4) is carried out in the presence of a catalyst.

17. The process according to claim 16, wherein the catalyst is an iodide, optionally potassium iodide.

18. The process according to claim 1, wherein (4) is carried out in a solvent.

19. The process according to claim 18, wherein the solvent is formic acid, acetic acid, propionic acid or a mixture thereof.

20. The process according to claim 1, wherein at least one compound (A) is selected from
compound of formula (XV), and
compound of formula (XVI).

21. The process according to claim 1, comprising (5) converting the fourth mixture from (4) to a fifth mixture by reducing an amount of one or more compounds (A) each by at least 50%, based on a starting amount of each compound (A) in the fourth mixture.

22. The process according to claim 21, wherein an amount of two or more compounds (A) is reduced.

23. The process according to claim 21, wherein at least one compound (A) is selected from
compound of formula (XV), and
compound of formula (XVI).

24. The process according to claim 21, wherein an amount of one or more compounds (A) is reduced in (5) by crystallization in a solvent.

25. The process according to claim 24, wherein the solvent is formic acid, acetic acid, propionic acid or a mixture thereof.

26. The process according to claim 24, wherein the amount of solvent is between 1 and 5 kilograms per kilogram of fourth mixture.

27. The process according to claim 24, wherein the crystallization is conducted at a temperature between 0 and 100° C.
